(19) United States
(12) Patent Application Publication
(10) Pub. No.: US 2026/0137351 A1
(43) Pub. Date: May 21, 2026

(54) MYOCARDIAL PERFORMANCE INDEX ESTIMATION BY USING NONINVASIVE DEVICES

(71) Applicants: LUCIAN EVDOCHIM, BRAILA (RO); AUGUSTIN SEMENESCU, BUCURESTI (RO); RUXANDRA VIDU, CITRUS HEIGHTS, CA (US); SILVIU MARCEL STANCIU, BUCURESTI (RO)

(72) Inventors: LUCIAN EVDOCHIM, BRAILA (RO); AUGUSTIN SEMENESCU, BUCURESTI (RO); RUXANDRA VIDU, CITRUS HEIGHTS, CA (US); SILVIU MARCEL STANCIU, BUCURESTI (RO)

(21) Appl. No.: 18/951,056

(22) Filed: Nov. 18, 2024

(51) Int. Cl. A61B 5/00; A61B 5/02; A61B 5/024; G16H 50/30

(52) U.S. Cl. CPC A61B 5/725; A61B 5/02028; A61B 5/024; A61B 5/7264; A61B 5/742; G16H 50/30

(57) ABSTRACT

A physiological signal processing method and apparatus are disclosed. The method involves extracting key waveform markers such as aspiration wave (ASW), diastolic point (DIA), systolic point (SYS), tidal wave (TDW) and dicrotic wave (DCW), which are associated with blood flow perturbations during blood circulation. These perturbations occurring within the systemic circulation originate from the heart's periodic mechanical activity, particularly from the left ventricle and right atrium. The extracted waveform markers are used to estimate individual waveform regions, including isovolumic contraction time (ICT), left ventricular ejection time (LVET), and isovolumic relaxation time (IRT). These parameters are further analyzed to compute the myocardial performance index (MPI). A validation step compares the computed parameters against real intervals to ensure the accuracy of the waveform regions and myocardial performance index. The validated results are output to the user, providing insights into cardiac performance, beneficial for both healthy individuals and patients with cardiovascular disorders.

### STATEMENT REGARDING FEDERALLY SPONSORED RESEARCH OR DEVELOPMENT

Not Applicable

### FIELD

Methods and apparatus consistent with exemplary embodiments relate to a signal processing method and apparatus, specifically for the automatic measurement of the myocardial performance index by noninvasively acquiring physiological signals from the systemic circulation.

### BACKGROUND OF THE INVENTION

Terms to be used in the invention:

The term “physiological signal(s)” as used herein refers to the physical quantities associated with blood flow or blood pressure phenomena, typically represented as a signal waveform. These signals are not limited to measurements from arterial or capillary vessels but encompasses blood flow dynamics across various types of blood vessels within the systemic circulatory system. The physiological signal can be acquired using a variety of techniques, including: electronics-based tensiometer acquisition technique using a pressure sensor with the associated physiological signal denoted as the arterial blood pressure waveform (ART); tonometry technique utilizing a pressure sensor with the associated physiological signal referred to as tonometry arterial waveform (TAW); skin displacement technique using a piezoelectric sensor, with the associated physiological signal referred to as piezoelectric-derived arterial waveform; radar technique using a radar transceiver-receiver sensor, with the associated physiological signal referred to as radar-derived arterial waveform; photoplethysmography (PPG) technique using an optoelectronic sensor with the associated physiological signal denoted as photoplethysmography waveform; remote photoplethysmography (rPPG) technique using a CMOS sensor with the associated physiological signal referred to as remote photoplethysmography waveform.

The term “waveform marker(s)” as used herein refers to a distinct fiducial point along the contour of a complete cardiac cycle waveform. These markers include aspiration wave (ASW), diastolic point (DIA), systolic point (SYS), tidal wave (TDW), and dicrotic wave (DCW).

The term “waveform region(s)” as used herein refers to specific time intervals within a complete cardiac cycle waveform, extracted based on the identified waveform markers. These regions include isovolumic contraction time (ICT), left ventricle ejection time (LVET), and isovolumic relaxation time (IRT).

The myocardial performance index (MPI), also known also as the Tei index, is a critical diagnostic parameter for evaluating cardiac performance. It is a derived by measuring the following components: isovolumic contraction time (ICT), left ventricular ejection time (LVET), isovolumic relaxation time (IRT), according to the formula:

$$MPI = \frac{ICT + IRT}{L V E T} \qquad ( 1 )$$

The current state of the art for assessing the MPI involves the use of Doppler echocardiography or tissue Doppler imaging to track the mechanical movement of the heart chambers. Typically, the MPI is evaluated for the left ventricular function, which is connected to the systemic circulation. By visualizing the heart's mechanical activity through Doppler technique, the MPI can be evaluated based on the acquired data, as disclosed in U.S. Pat. No. 10,357,228.

The individual components of the MPI, i.e. ICT, IRT, LVET are also valuable diagnostic tools on their own. For example, a prolonged ICT may indicate a weakening of the left ventricle smooth muscle, impairing its ability to properly inject blood into the systemic circulation. Similarly, a shortened LVET, normalized to the current heart rate, may suggest impaired left ventricular contraction, which could be associated with reduced cardiac output. These components serve as valuable markers for detecting deterioration of myocardial function, potentially leading to heart failure events.

In the context of high-value diagnostic tools, a major drawback of MPI is the bulkiness of the Doppler-based device typically used, which limits accessibility and makes myocardial evaluation available only during scheduled office visits with a trained clinician. As a result, the number of MPI measurements is limited, typically taken during a single office visit rather than throughout the day. A comprehensive assessment would involve continuous acquisition of MPI and its component parameters over a full day.

The increasing popularity of wearable devices capable of acquiring physiological signals offers a potential solution to this issue. However, to determine whether these devices can extract MPI from the systemic circulation, it is important to evaluate their ability to capture the necessary information. The paper titled “The phases of the cardiac cycle” by Aldo et al. published in American Heart Journal 1972, Vol. 83, No. 5, pages 705-711, describes how the left ventricular mechanic activity is synchronized with a progression of the entire cardiac cycle, as captured by various physiological signals. In this study, the arterial pressure waveform contour is used as a representative physiological signal, although specific waveform components are not identified. Instead, the cardiac cycle's phases are delimited using heart sounds and electrocardiogram (ECG) data. This approach is an enhancement of Wiggers's diagram, which first described the mechanical phases of the heart.

The reinterpretations of Wiggers diagram in the scientific literature have allocated specific waveform components to define the waveform regions within the physiological signal, without rigorous validation. US patents US20240065567A1, US20110190601A1 and U.S. Pat. No. 11,666,230 describe how left ventricular ejection time (LVET) is delimited from the diastolic point (DIA), marking the start of the cardiac cycle, to the onset of the dicrotic waveform (DCW), also known also as incisura or dicrotic notch. Additionally, US20100228136A1 also uses the DCW onset as the boundary between LVET and the beginning of the isovolumic relaxation time (IRT) phase.

A novel interpretation of physiological signal waveform components is disclosed in “Elastic Water Hammer Effect in Arterial Network: Origin of the Tidal and the Dicrotic Wave in Blood Pressure Waveforms” by Evdochim et al. published in Conf. Proc. IEEE USBEREIT 2024, pages 8-11. The study suggests that the cessation phase of LVET triggers a blood flow perturbation within the systemic circulation due to the rapid closure of semilunar valves, which generates the prime waveform component, also called tidal wave (TDW) that is a detectable physiological signal. The magnitude of TDW and the detection ability within the acquired physiological signal is function of hemodynamic state of the user and the detection capability of the used apparatus. Shortly after, a second blood flow perturbation occurs that forms the second waveform component, which is the so-popular dicrotic wave. This blood flow perturbation originates from the rapid contraction of the right atrium that is blocking the incoming blood flow. The magnitude of the DCW is typically greater than that of the TDW due to the higher energy associated with the right atrium's contraction that halts the blood flow.

The feasibility of using the LVET as a marker for estimating TDW waveform component, instead of the commonly used DCW, is discloses by “Left Ventricular Ejection Time Estimation from Blood Pressure and Photoplethysmography Signals Based on Tidal Wave” by Evdochim et al. published in J. Appl. Sci. 2023, Vol. 13, No. 19:11025. The study shows the capability of wearable devices represented by the photoplethysmography technology to extract LVET estimation from the physiological signal, which produces lower errors compared to using DCW. Furthermore, it discusses the delay in the observed blood flow perturbation in the physiological signal due to waveform traveling effects through real arterial system. This delay is influenced by factors such as vasodilation and vasoconstriction. Therefore, the true cessation point of LVET is represented by TDW, despite the literature's traditional use of DCW.

In “Group Delay Effect Analysis Between Arterial Blood Pressure and Photoplethysmography Waveforms” by Evdochim et al. published in IMFBE Proc. EHB 2023, Vol. 109, pages 30-38, the validation of the delay time between TDW and DCW in physiological signal is disclosed. This study discloses an average delay between 5 ms and 25 ms, which can spike up to 50 ms under low arterial blood pressure conditions.

Another study, “Coronary-aortic interaction during ventricular isovolumic contraction” by Marc J van Houwelingen et al. published in J. Med. Biol. Eng. Comput. 2011 Vol. 49, pages: 917-924, identifies the existence of a pre-systolic perturbation within blood pressure waveform occurring before the DIA point. The duration of the reported perturbation within the physiological signal is comparable with the isovolumic contraction duration (ICT). However, the origin of this blood flow perturbation, proposed as related to coronary network modulation, was not clearly linked to the coronary network itself.

The invention disclosed herein addresses these challenges by enabling the estimation of MPI from noninvasive physiological signals, eliminating the bulkiness of Doppler devices. The signal processing methods and apparatus for estimating MPI from physiological signals are not limited to wearable devices, offering broader applicability.

Existing literature already reports the real intervals for MPI and its components (ICT, LVET, IRT) from Doppler assessments in various cardiovascular disease (CVD) investigations. For example, ICT ranges between 10 ms and 120 ms, LVET between 150 ms and 450 ms (strongly influenced by heart rate), and IRT between 10 ms and 200 ms. The MPI itself typically ranges from 0.3 to 1.0.

By expanding the capabilities of noninvasive technologies to include Myocardial Performance Index (MPI) measurement the proposed method offers several notable benefits. Continuous, real-time monitoring of MPI allows for a more comprehensive view of a patient's cardiovascular health over time, enabling early detection of myocardial performance deterioration. This continuous detection can lead to timely interventions before symptoms worsen. The availability of these parameters, i.e. MPI and its constituent parameters, ICT, IRT and LVET, to both users and clinicians optimizes healthcare resources, reducing the need for frequent in-person visits and enabling healthcare providers to prioritize critical cases. Moreover, this integration enhances accessibility to medical care, particularly for patients with limited mobility or those in remote locations.

### SUMMARY OF THE INVENTION

The present invention addresses the primary drawback of traditional MPI (Myocardial Performance Index) measurements, which require bulky Doppler equipment. By enabling MPI estimation from physiological signals, this invention allows the integration of MPI assessment into wearable devices, significantly improving accessibility for users and facilitating continuous health tracking. This approach eliminates the need for periodic office visits with clinicians, reducing the burden on medical personnel and enabling them to prioritize critical cases. It also allows healthcare providers to remotely monitor a patient's heart performance, optimizing the use of medical resources.

A key advantage of embedding MPI measurement into wearable devices is the ability to perform multiple measurements throughout the day. This allows users to track their heart performance in various real-world scenarios, such as during periods of rest, stress, physical activity, or sleep. With this functionality, MPI estimation based on physiological signals provides a more comprehensive view of a user's cardiac health. Users can measure MPI on-demand, such as when they experience chest pain, or set the device to automatically record during specific activities.

The signal processing method and apparatus may also consider additional factors related to the acquired physiological signals, such as the sampling frequency, any pre-filtering steps applied, the type of physiological signal being measured, the amplification or attenuation of the signal, and the sensitivity range of the sensor. This comprehensive approach ensures the accuracy and relevance of the measurements.

In some embodiments, the signal processing method may also incorporate data from an external accelerometer sensor to account for user movement during signal acquisition. If the acquired physiological signal is affected by movement artifacts, such as sensor displacement or low signal quality, the system can reject the erroneous data to prevent inaccuracies in MPI estimation.

The presented invention may include an electronic front-end interface to process the acquired physiological signal. This front-end can perform various signal processing steps, including filtering (using low-pass and high-pass filters), amplification, or attenuation of the signal to ensure it is in an optimal form for analysis.

Additionally, the physiological signal may be converted from analog to digital format using an analog-to-digital converter (ADC). Once in digital form, the signal is subject to further processing, including digital filtering, amplification, or attenuation, to enhance the quality of the data.

To detect at least one valid cardiac cycle, the signal processing method may analyze the acquired physiological signal values. If multiple cardiac cycles are detected, the system can compute the corresponding heart rate.

According to another aspect of the exemplary embodiment, the detected cardiac cycle could be rejected if it is affected by the acquisition artifacts such as: sensor motion with respect to the measurement site of the inputted physiological signal, poor signal to noise ratio due to improper physiological signal processing procedure, poor signal to noise ratio due to low performance acquisition of the involved physiologic sensor. For example, if the computed heart rate is outside the expected range, the cycle may be discarded.

The signal processing method may also include detecting key waveform components associated with the cardiac cycle, such as aspiration wave (ASW), diastolic point (DIA), systolic point (SYS), tidal wave (TDW), and dicrotic wave (DCW). These components can be identified using logic rules or mathematical operations, such as detecting local maxima or minima along the waveform. In some cases, a pivot point, such as the systolic component, may be used to search for other waveform components within a defined time window.

According to another aspect of the exemplary embodiment, if the quality of the acquired signal is low, some waveform components may not be detected. This could be due to factors such as poor signal-to-noise ratio or limited sensor sensitivity. In such cases, the signal processing method may exclude the undetected components from the MPI calculation.

In addition to MPI, the signal processing method can compute the individual waveform regions (e.g., Isovolumic Contraction Time (ICT), Left Ventricular Ejection Time (LVET), and Isovolumic Relaxation Time (IRT)) based on the valid waveform components detected. If only partial waveform regions are detected, the system can still compute available parameters without final MPI estimation, providing useful insights based on the data at hand.

To ensure the validity of the calculated waveform regions and MPI, the signal processing method may include checking the validity of individual waveform regions time duration and MPI parameter based on the reported real values interval. The method may further include, inputting user personalized waveform regions time duration interval for an increased accuracy.

According to another aspect of the exemplary embodiment, the individual waveform regions could be rejected if the values measured time duration are outside the known real-world intervals or user-specific personalized ranges. If any measured duration falls outside the acceptable boundaries, those waveform regions can be rejected.

The system may output standalone MPI values, individual waveform region durations, or a combination of both, depending on the user's needs. These results can also be paired with the corresponding heart rate value for more context. Additionally, the results can be stored in memory for future analysis, with users able to query the stored data on demand or have it retrieved automatically.

By integrating these signal processing techniques into wearable devices, the present invention offers a more accessible, continuous, and comprehensive method for tracking myocardial performance, allowing users to monitor their heart health more effectively and providing valuable data to clinicians for better-informed decision-making.

### BRIEF DESCRIPTION OF THE DRAWINGS

The above and other features of the exemplary embodiments will become more apparent by describing in detail exemplary embodiments thereof with reference to the attached drawings in which:

FIG. 1 is an illustration of heart anatomy by highlighting the biological elements representative for systemic circulation.

FIG. 2 is an illustration of the systemic circulation upper and lower regions divided by the heart location plane.

FIG. 3 is a diagram illustrating the correspondence between heart mechanical phases involved in systemic circulation, physiological signal waveform's components and waveform regions.

FIG. 4A is an example of a physiological signal acquired by mechanical fashion using a pressure sensor.

FIG. 4B is an example of a physiological signal acquired by optoelectronic fashion using a photoplethysmography sensor.

FIG. 5 is a block diagram of a signal processing apparatus according to an exemplary embodiment.

FIG. 6 is a flowchart illustrating the signal processing method for MPI estimation based on the inputted physiological signal.

### DETAILED DESCRIPTION OF THE INVENTION

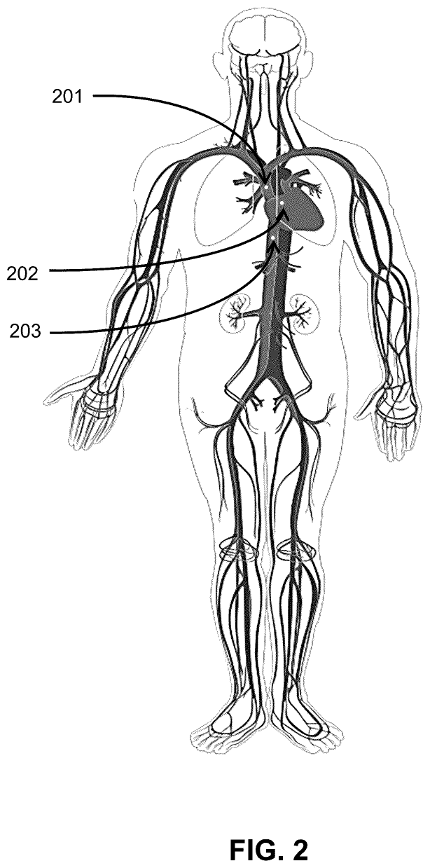

The estimation of the MPI parameter from the physiological signal is based on the heart's 202 mechanical activity and its influence to the blood flow evolution inside the upper part of systemic circulation 201 and lower part of the systemic circulation 203 according to FIG. 2.

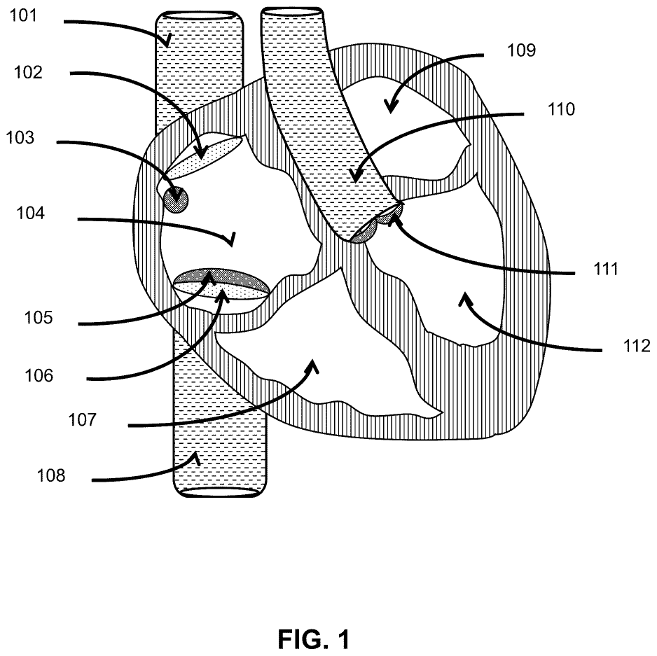

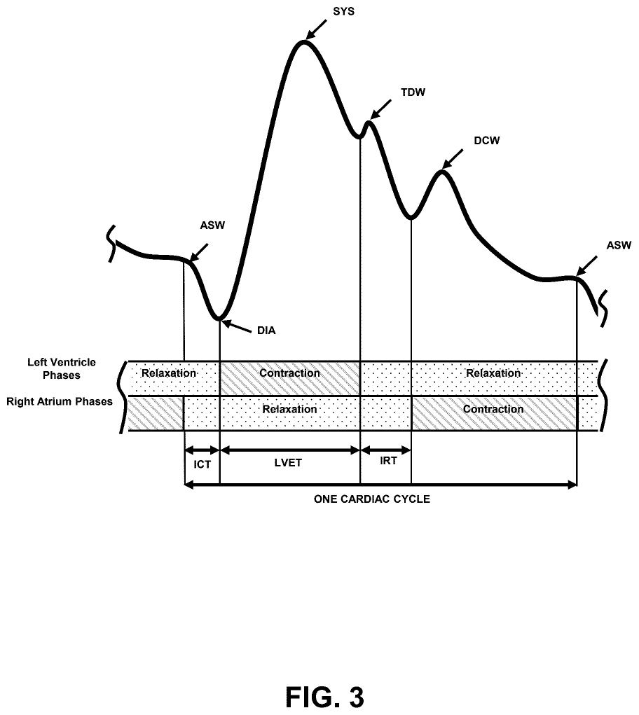

The primary parameter that compounds the MPI is left ventricular ejection time (LVET). According to the FIG. 1 this cardiac phase of the left ventricle (LV) 112 is delimited from diastolic point (DIA) until to the onset of first physiological signal disturbance that occurs after systolic point (SYS), as shown in FIG. 3. This disturbance called TDW origins from the rapid closure of semilunar valves (SV) 111 that disconnect LV chamber 112 from the rest of the systemic circulation 201 and 203 that is starting with ascending aorta 110 according to FIG. 1.

During this rapid transition of SV 111 closure, the negative Water Hammer (WH) effect is occurring being characterized by the appearance of locally negative pressure zone in the vicinity of interruption. In this case, the affect zone of negative WH effect is the beginning part of ascending aorta 110. Since the human's blood vessels have elastic properties, the tendency of negative zone occurrence is rapidly compensated by the vessels' rebound effect. In this case, the closer ascending aorta' segment 110 next to the SV 111 will rebound inside. As result, this locally rebounding phase is additionally pushing the blood mass though the systemic circulation 201 and 203 giving a supplementary gain in the blood flow. In other words, the aortic segment 110 inside rebound phenomenon is equivalent of a newer LV ejection phase but with a smaller magnitude.

The second parameter that compounds MPI is the isovolumic relaxation time (IRT). According to FIG. 3 this cardiac phase is delimited from the onset of TDW until to the second physiological signal disturbance. The second disturbance called DCW origins from the rapid contraction of the right atrium (RA) 104 that is acting as a blood collector chamber. During this rapid transition, the positive WH effect, known as Joukousky effect, is characterized by the appearance of locally positive pressure zone near RA 104 element.

For the ending of the upper part of systemic circulation 201 the interface, superior cavoatrial junction (SCJ) 102, between RA 104 and superior vena cava (SVC) 101 is a one region of DCW formation. The rapid blocking and releasing of SCJ 102 is controlled by the electrical activity of sinoatrial node (SN) 103. At the moment of rapid SCJ 102 closures, the blood flow direction from SVC 101 to RA 104 is sudden stopped. According to WH effect, all the mechanical inertia of the blood flow shall dissipate in the surrounding area by creating a flow disturbance. This perturbation firstly will create in turn an additional blood pressure quantity on the vessel's walls what belong to the ending part of upper systemic circulation 201. Due to physical phenomenon of waveform travelling, the perturbation will travel along of the entire upper part of systemic circulation 201.

For the lower part of systemic circulation 203 the interface between RA 104 and inferior vena cava 108 (IVC), called inferior cavoatrial junction 106 (ICJ), is another region of DCW formation. Herein, the mechanism is different: the electrical activity triggered by SN 103 will spread inside RA 104 and triggers the entire chamber contraction. As effect, upon the already collected blood mass the RA 104 contraction will exert a mechanical force. From physical point of view, the collected blood mass will be pushing in turn in all direction. Thus, will be pushing effect even on the ICJ 106 since SCJ 102 is already blocked by SN 103 activation. The naturally back-flow that would occur in ICV 108 is blocked by the Eustachian valve 105 that is acting as a one-direction valve element. As long as the RA 104 contraction process is rapidly in terms of milliseconds, the blood that is flowing from IVC 108 will encounter a rapid stop. The same positive WH mechanism as in SCJ 102 case will be present. Thus, a second DCW will be formed in the lower part of systemic circulation 203. This perturbation waveform will be traveling along the entire lower part of systemic circulation 203.

According to the heart physiology FIG. 1 not only RA 104 will be contracting but its complementary left atrium (LA) 109 chamber. This process is meant to push the blood mass from RA 103 and LA 109 into the right ventricle 107 (RV), respectively LV 112 ventricle ones. The starting phase of receiving blood mass from the LA 109 part will create inside LV 112 physical pressure. Between the onset of TDW, triggered by SV 111 closure and the onset of receiving blood mass from LA 109, the LV 112 cardiac muscle is in a relaxing state. Thus, the IRT is measured from the TDW onset, associated with LVET ceasing period, until to DCW onset associated with LV 112 blood filling process, FIG. 3.

The third parameter that compounds MPI is the isovolumic contraction time (IVC). According to FIG. 3 this cardiac phase is delimited from the onset of ASW landmark until to the DIA point. After the contraction of RA 104 the cardiac smooth muscle fibers are transiting into a relaxation state. This rapidly transition marks the availability of RA 104 chamber to start acting again as a blood collector element. Thus, the blood mass temporarily interrupted from RA 104 by SCJ 102 and ICJ 106 begins to be aspirated into the heart chamber. As result, this rapid transition toward aspiration phase creates a blood flow disturbance in the both sides of systemic circulation 201 and 203.

In parallel, LA 109 is following the same RA 104 pattern. As a result, the LV 112 stops to receiving blood mass and begins a new contractile state. This transition of LV 112 cardiac muscle activity coincides with the onset of ASW formation FIG. 3. Inside the LV's 112 cardiac muscle contractile force is building up until the physical force overcomes blood pressure inside ascending aorta 110 by opening SV 111. The beginning of LVET FIG. 3 is marked by the presence of DIA point. Thus, ICT is measured from the onset of ASW until the DIA according to FIG. 3.

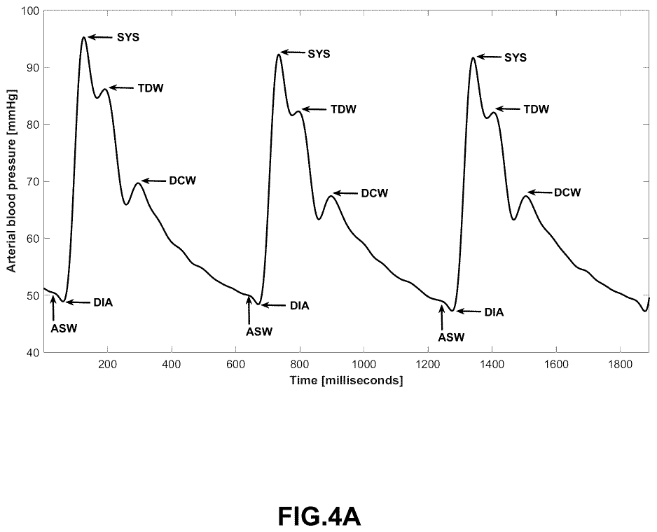

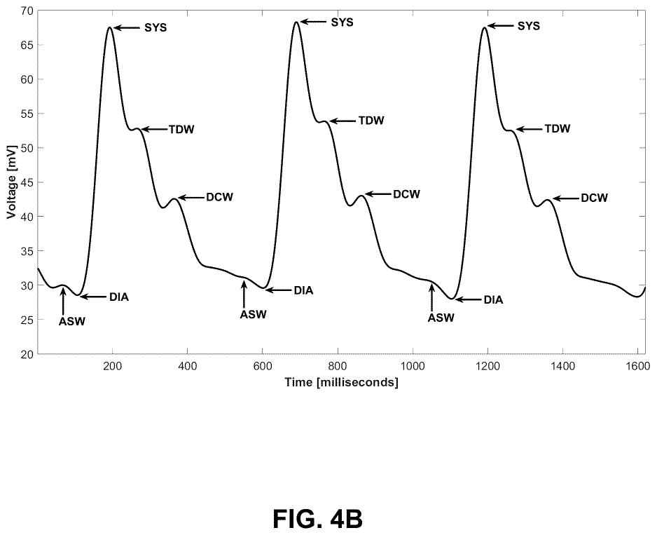

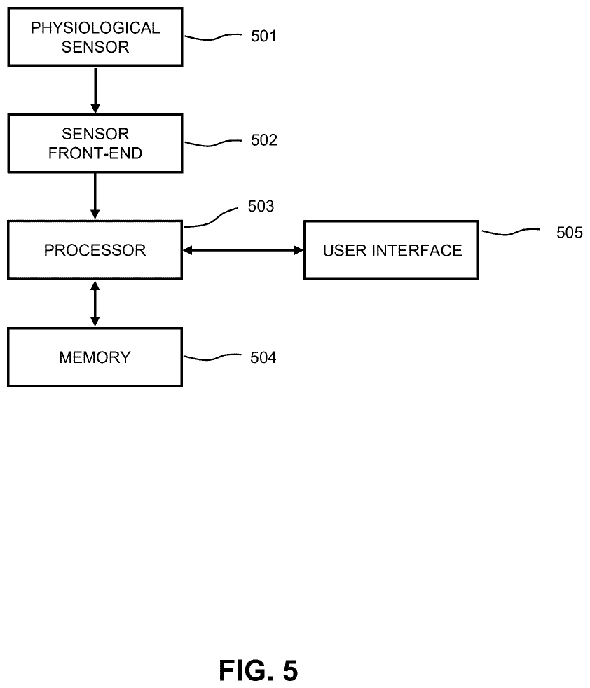

The apparatus that process the physiological signal by detecting waveform markers is depicted in FIG. 5 containing at least the following blocks: physiological sensor 501, sensor front-end 502, processor 503, memory 504 and user interface 505. The physiological signal acquired by the physiological sensor 501 could have the specific waveform morphology illustrated in FIG. 4A and FIG. 4B.

In another implementation the apparatus could contain at least one physiological sensor 501, processor 503, memory 504 and user interface 505, thus excluding the sensor front-end 502. Further, in another embodiment the memory 504 could be excluded if the apparatus provides the current results to only an user.

The physiological sensor 501 acquires the physiological signal according to the defined term description. Usually, the physiological sensor 501 converts the physiological signal into an analog signal format. In another embodiment the physiological sensor 501 coverts the physiological signal into an digital signal format.

In this invention, the physiological sensor 501 triggers the acquisition process of the physiological signal at the request of the processor 503 instructions. The acquisition procedure could be continuous or intermittent at a preconfigured times intervals defined by the processor 503 instructions. In another embodiment, physiological sensor 501 could receive additional information from an external motion sensor to start or to halt the physiological signal acquisition phase.

The physiological signal acquired from the physiological sensor 501 is transmitted to the sensor front-end 502. The primary function of the sensor front-end 502 is to modify the electrical properties of the converted physiological signal. These modifications performed by the sensor front-end 502 can include, but are not limited to, amplification, attenuation, or filtering the electrical quantity of the converted physiological signal. In some embodiments, the sensor front-end 502 may also convert the analog quantity of the physiological signal into a digital format, typically using an analog-to-digital converter apparatus.

In this invention, the sensor front-end 502 can be controlled by the processor 503 to perform various modifications to the electrical quantity of the physiological signal signal, enabling flexible processing of the physiological data.

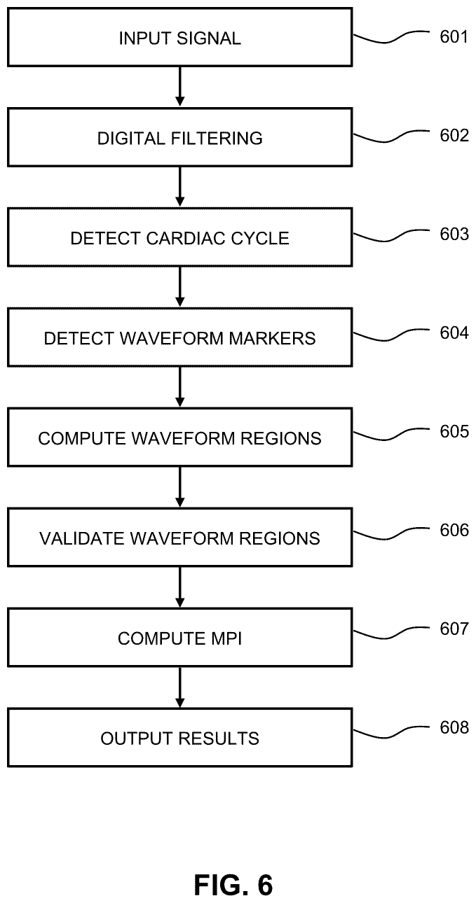

The processor 503 is primary responsible for implementing the signal processing method outlined in the flowchart of FIG. 6 which involves processing the digital version of the acquired physiological signal. For example, the electric quantity acquired by the physiological signal shown in FIG. 3 is interpreted by the method in FIG. 6, whether it originates from sources such as the arterial blood pressure waveform in FIG. 4A or the electrical voltage waveform in FIG. 4B. In another example, the processor 503 may store the electrical quantity of the physiological signal in the memory 504 for later signal analysis.

The processor 503 also communicates bidirectionally with the user interface 505, allowing user interaction. The invention takes into consideration that the signal processing method in FIG. 6, executed by the processor, may be initiated by a user request through the user interface 505. Additionally, the system may be configured to automatically begin the signal processing method FIG. 6 at predefined time intervals or specific moments, according to user preferences.

The physiological signal processing method in FIG. 6 is executed by the processor 503. However, the invention also contemplates that the processor 503 will pause execution of the signal processing method in FIG. 6 if an external motion sensor detects user movement. In such cases, the signal processing may be halted to avoid introducing artifacts or errors into the physiological signal caused by movement.

The physiological signal processing method outline in FIG. 6 begins with the inputting physiological signal operation 601, where the signal is received in digital format. During this operation 601 a plurality of samples of the digitally converted physiological signal are processed. Alternatively, operation 601 may receive a plurality of values from the memory 504, which were previously stored by the processor 503 under its instructions.

During the signal operation 601, the maximum number of digitally converted samples that can be input is configurable either by the processor 503's instructions or manually by the user through the user interface 505.

After operation 601, the received plurality of values are moved to the digital filtering operation 602 under the control of the processor 503 instruction. In operation 602, the plurality of values are modified using a digital filtering procedure, which may include, but is not limited to, low-pass filtering, high-pass filtering, or a combination of both methods. The main objective of this digital filtering operation 602 is to remove electronic noise added to the physiological signal acquired from the apparatus shown in FIG. 5. To execute operation 602, the processor 503 instructions requires information about the sampling frequency used during the physiological signal acquisition from the apparatus in FIG. 5.

In some implementations, the sampling frequency value can be provided to the processor 503 by the individual physiological sensor 501, the sensor front-end 502, or through the user interface 505. Based on the provided sampling frequency, the digital filtering operation 602 is carried out accordingly by the involved apparatus FIG. 5.

Following operation 602, the filtered plurality of values are passed to the cardiac cycle detection operation 603. The primary goal of operation 603 is to identify a valid cardiac cycle period from the provided plurality of values. This operation may involve logic rules, mathematical functions, or a combination of both. In one embodiment, the cardiac cycle is considered valid if a local maximum associated with the SYS component (shown in FIG. 3, FIG. 4A, and FIG. 4B) is detected. Alternatively, in another embodiment, operation 603 may apply a low-pass filtering mathematical function with a cutoff frequency between 1 Hz and 3 Hz to enhance the detection of the SYS component (shown in FIG. 3, FIG. 4A, and FIG. 4B).

Additionally, operation 603 may use the Fast Fourier Transform (FFT) technique on the filtered plurality of values to confirm the presence of a valid cardiac cycle.

The operation 603 can detect one or more valid cardiac cycles, or none at all, based on the plurality of values processed from the digital filtering operation 602. If no valid cardiac cycle is detected, the processor 503 instructions halt further execution of the method outlined in FIG. 6. The processor 503 instructions then returns to operation 601 to wait for the next set of physiological signal values.

In another embodiment, when operation 603 detects at least two valid cardiac cycle, the processor 503 instructions calculate the heart rate by determining the time difference between the detected cardiac cycles. If the computed heart rate falls outside a valid range, the processor 503 halts the execution of the method in FIG. 6 and is returning to operation 601.

Additionally, operation 603 may further assess the validity of the detected cardiac cycles by evaluating the consistency of the SYS and DIA waveform markers (as shown in FIG. 3, FIG. 4A and FIG. 4B).

After operation 603 successfully detects and validates at least one cardiac cycle, the processor 503 instructions passes the plurality of values to operation 604. In this operation, the waveform markers (as shown in FIG. 3) are detected using methods such as logic rules, mathematical functions, or a combination of both. In one embodiment, the local maximum point between the SYS and DIA waveform markers (FIG. 4B) is interpreted as the DCW, as it represents the second significant waveform marker after the SYS point due to its comparable signal energy. In another embodiment, the presence of two local maximum points between SYS and DIA (FIG. 3 and FIG. 4A) is interpreted as the TDW and DCW, reflecting cardiac cycle consistency.

In another embodiment, the detection of the waveform components in operation 604 may rely on searching for the local maximum points within a defined time frame relative to a chosen pivot point. For example, if the DIA point is used as the pivot point, the ASW waveform marker should occur within 120 ms prior to it, which corresponds to the maximum duration of the real time of ICT. In another embodiment, when the DIA point is used as the pivot point, the TDW waveform marker should occur within 450 ms afterward, covering the maximum duration of LVET and accounting for the SYS point (FIG. 3).

In another embodiment, operation 604 may detect all, some, or none of the waveform markers. If none of the waveform markers are detected, the processor 503 halts further execution of the method in FIG. 6 and returns to operation 601 to wait for a new set of physiological signal values.

In another embodiment, if only some of the waveform markers are detected, the processor 503 instruction may continue executing the method from operation 604, provided that the detected markers are sufficient to compute partial waveform regions. For example, the presence of only ASW and DIA waveform markers may be enough to compute the ICT duration, as shown in FIG. 3. Similarly, the presence of DIA and TDW waveform markers may be sufficient to compute the LVET duration.

Once operation 604 detects a suitable combination of waveform markers to compute at least one waveform region, the processor 503 instruction moves to operation 605 to calculate the waveform regions. The calculation of these waveform regions depends on the required waveform markers: for ICT, ASW and DIA are needed; for LVET, DIA and TDW are required; and for IRT, TDW and DCW are required, as indicated in FIG. 3.

After operation 605, where at least one waveform region has been computed, the processor 503 proceeds to operation 606 to validate the waveform region. In this step, the computed time durations of the waveform regions are checked against established real-world intervals, as reported in studies and literature. For example, the ICT duration should fall within the range of 10 ms to 120 ms, the LVET duration should range from 150 ms to 450 ms, and the IRT duration should be between 10 ms and 200 ms.

In some embodiments, operation 606 may also use statistical methods to further validate the waveform region durations, including the occurrence of certain combinations of values. For example, an LVET duration of 200 ms is more likely to be accompanied by a narrow ICT range (50 ms-100 ms) and a narrow IRT range (100 ms-180 ms).

Moreover, operation 606 could incorporate personalized waveform region duration intervals based on the user's hemodynamic profile. For instance, a user could perform a Doppler echocardiogram in a medical office to define their personalized waveform region intervals. After a clinical examination, the user might have an LVET between 200 ms and 300 ms, ICT between 80 ms and 100 ms, and IRT between 100 ms and 120 ms. These personalized waveform regions time intervals can be input into the processor 503 instruction via the user interface 505 and used as a validation method in operation 606.

Once operation 606 step is complete and all waveform regions have been validated, the processor 503 instructions will proceed to compute the MPI in operation 607, using Equation (1), provided all waveform regions are valid. The MPI result will be checked against real-world values, with a valid range typically between 0.3 and 1 unit.

If only some of the waveform regions are validated, the processor 503 instruction may skip the MPI computation and proceed directly to the result output in operation 608.

Operation 607 may also use statistical methods to validate the MPI result, based on the user's profile. For instance, depending on the user's age and medical history, the MPI may be more likely to fall within a narrower range, such as 0.5 to 0.7 units. In another embodiment, the user might manually input predefined MPI intervals based on clinical findings. These personalized values can be entered into the processor 503 instructions via the user interface 505.

If the MPI result falls outside the valid range in operation 607, the processor 503 instruction may halt the execution of method in FIG. 6 and prevent further execution.

After completing operation 607, the processor 503 instructions proceeds to operation 608, where the results are output. Here, the validated waveform regions' time durations, including the MPI, are paired with the time of physiological signal acquisition. In another embodiment, if multiple waveform regions and MPI results are available, the operation 608 may also pair these with the heart rate result computed in operation 603.

In operation 608, the results are displayed to the user via the user interface 505. In another embodiment, if no results are available, the user interface 505 may show an error message. Additionally, the results of operation 608 can be stored in memory 504 by the processor 503 instructions, and in some cases, the user interface 505 can later display the results from memory 504. In this embodiment, the user can choose to display the results manually or automatically from memory 504 at a later time.

1. A signal processing apparatus, comprising: a physiological sensor, a sensor front-end, a memory, and a processor configured to execute the instructions to:
receive waveform samples into a digital format from at least one physiological sensor;
apply digital filtering to the plurality of values of the physiological signal in digital format;
detect and validate the cardiac cycle from the plurality of values of the filtered digital signal;
further detect and validate waveform markers, including ASW, DIA, SYS, TDW and DCW from the plurality of values by identifying the portions of the physiological signal with corresponding activity;
compute and validate waveform regions including IRT, LVET and IRT by analyzing the corresponding waveform markers;
compute and validate an estimation of MPI parameter by analyzing the waveform regions results including ICT, LVET, IRT;
pair the validated waveform region results with the estimated MPI parameter and associated heart rate value;
output the validated waveform region results including ICT, LVET and IRT;
output the validated results of the MPI estimated parameter;
output the validated pairing results of waveform regions, MPI estimated parameter and associated heart rate value.

2. The signal processing apparatus of claim 1, wherein the processor is configured to receive physiological signal samples from the physiological sensor at a predefined sampling frequency.

3. The signal processing apparatus of claim 1, wherein the processor is further configured to receive waveform samples from sensor front-end at a predefined sampling frequency.

4. The signal processing apparatus of claim 1, wherein the processor is further configured to initiate the physiological signal acquisition process based on predefined user settings.

5. The signal processing apparatus of claim 1, wherein the user interface allows for input of personalized waveform regions and MPI intervals by the user.

6. The signal processing apparatus of claim 1, wherein the waveform regions and MPI estimated results stored into the memory can be queried by the user.

7. A method for processing physiological signals obtained from physiological sensors, comprising the operations of:
receiving waveform samples in digital format from at least one physiological sensor;
applying digital filtering to the plurality of received physiological signal samples;
detecting and validating the cardiac cycle from the plurality of values of filtered signal;
detecting and validating waveform markers, including ASW, DIA, SYS, TDW and DCW from the plurality of received values, by identifying the portions of the physiological signal with corresponding activity;
computing and validating waveform regions, including IRT, LVET and IRT, based on detected waveform markers;
estimating and validating the MPI parameter by analyzing the computed waveform region results, including ICT, LVET, IRT;
pairing the validated the waveform region results with the MPI estimate and the associated heart rate value;
outputting the validated waveform regions results, including ICT, LVET and IRT;
outputting the validated MPI parameter estimate; and
outputting the validated pairings of waveform regions, the MPI parameter estimate, and the associated heart rate value.

8. The method of claim 7, wherein the operation of receiving physiological signal' samples in digital format includes receiving the sampling rate information from the physiological sensor in use.

9. The method of claim 7, wherein the operation of receiving physiological signal samples further involves storing the samples and the corresponding detected and validated cardiac cycle data for future analysis.

10. The method of claim 7, wherein the operation of digital filtering involves applying a combination of low-pass and high-pass filtering techniques at a predefined cutoff frequency.

11. The method of claim 7, wherein the operation of detecting and validating the cardiac cycle includes rejecting certain signal values, which may lead to halting the signal processing method.

12. The method of claim 7, wherein the operation of detecting and validating the cardiac cycle further includes storing the validated plurality of the signal values into a memory for future analysis.

13. The method of claim 7, wherein the operation of detecting and validating the cardiac cycle further includes computing the heart rate if at least two cardiac cycles are validated.

14. The method of claim 7, wherein the operation of detecting and validating the cardiac cycle further involves using logic rules, mathematic functions, or a combination of both.

15. The method of claim 7, wherein the operation of detecting and validating the waveform markers involves using logic rules, mathematic functions or a combination of both.

16. The method of claim 7, wherein the operation of computing and validating the waveform regions involves comparing the results against historical data, reference ranges, user-specific ranges, or a combination thereof for accuracy.

17. The method of claim 7, wherein the operation of computing and validating the waveform regions further involves storing the validated results in a memory for future analysis.

18. The method of claim 7, wherein the operation of computing the MPI parameter involves comparing the results against historical data, reference ranges, user-specific ranges or a combination thereof for accuracy.

19. The method of claim 7, wherein the operation of computing MPI parameter further involves storing the validated results into a memory for future analysis.

20. The method of claim 7, wherein the operation of outputting the validated waveform regions results involves displaying the available waveform regions and the estimated MPI results, or partially displaying them to the user.

21. The method of claim 7, wherein the operation of outputting the validated waveform region and MPI results further involves pairing with the associated heart rate value and the time moment of the acquisition phase.
